(19) United States
(12) Patent Application Publication
(10) Pub. No.: US 2006/0002882 A1
(43) Pub. Date: Jan. 5, 2006

(54) Rinse-out cosmetic composition comprising elastomeric film-forming polymers, use thereof for conditioning keratin materials

(76) Inventors: Isabelle Rollat-Corvol, Paris (FR); Jonathan Gawtrey, Boulogne (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/171,315

(22) Filed: Jul. 1, 2005

Related U.S. Application Data
(60) Provisional application No. 60/587,853, filed on Jul. 15, 2004.

(30) Foreign Application Priority Data
Jul. 1, 2004 (FR) 04 07305

(51) Int. Cl. A61K 8/87; A61K 8/81

(52) U.S. Cl. 424/70.16; 424/70.17

(57) ABSTRACT

The present disclosure relates to a cosmetic composition comprising, in a cosmetically acceptable medium, at least one elastomeric film-forming polymer, wherein film obtained by drying the at least one elastomeric film-forming polymer at ambient temperature and at a relative humidity of 55%±5%, has properties comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%, the at least one elastomeric film-forming polymer being water-soluble. This composition makes it possible, for example, to condition keratin fibers such as the hair.

This application claims the benefit of U.S. Provisional Application No. 60/587,853, filed Jul. 15, 2004, the contents of which are incorporated herein by reference. This application also claims the benefit of priority under 35 U.S.C. § 119 to French Patent Application No. 04 07305, filed Jul. 1, 2004, the contents of which are also incorporated by reference.

The present disclosure relates to a cosmetic composition comprising, in a cosmetically acceptable medium, a film-forming polymer with defined elastomeric characteristics. It also relates to the use of this composition for formulating rinse-out products intended for human keratin materials such as the skin, the nails and keratin fibers, these products being, for example, cleansing or makeup-removing products for the hair, the skin or the eyelashes, such as bath products, makeup-removing products, shampoos and conditioners for the hair, for example, conditioning and/or styling agents.

The present disclosure also relates to a method for conditioning, shaping or maintaining keratin fibers, such as the hair, using this composition.

Setting of the hairstyle is an important element of hair styling, and involves the maintaining of a shape that has already been formed. According to the present disclosure, the term “styling composition” concerns any type of hair composition that can be used to perform the styling.

Among the hair products for setting hair that are most widely available on the cosmetics market, mention may be made of spray compositions in the form of an aerosol or in a pump-action bottle, such as lacquers, sprays or mousses. These products can be comprised of a solution, most commonly alcoholic or aqueous-alcoholic, and a film-forming polymer that is water-soluble or soluble in alcohol, mixed with various cosmetic adjuvants, or alternatively products to be applied by hand, such as gels and waxes.

However, these hair formulations for styling, and especially aerosol sprays and lacquers, do not yet allow the hairstyle to satisfactorily withstand the various natural movements that occur in daily life, such as walking, head movements or gusts of wind. In addition, these compositions give the head of hair a feeling of stiffness, called “helmet effect”. Styling shampoos that have the advantage of combining both washing of the hair while at the same time allowing it to be shaped during drying are also known as styling products, which allows users, who are increasingly busy, to gain time, and has the advantage of not keeping compressed gas products at their home, which products can, under certain conditions of incorrect use, explode and cause damage, such as burning the users.

The polymers conventionally used for formulating hair products are anionic, amphoteric or non-ionic film-forming polymers that result in the formation of films having a more or less hard and brittle nature.

When the polymer is too brittle, the percentage elongation at break measured on the film is low, i.e. generally less than 2%, and the hairstyle does not hold over time.

To remedy this problem, these polymers have already been mixed with plasticizers and coatings that are more flexible and not friable. However, these films are deformable and plastic, i.e., after deformation, they only very slightly recover their initial shape. Although the hold of the hairstyle is improved, it is not yet satisfactory since the shape of the hairstyle changes over time.

Moreover, many styling compositions that exist at the current time have the same drawback: they are not intended to allow the hairstyle to be subsequently modified to a desired shape, which is other than that initially formed, without recommencing once again the styling and setting operations. In addition, under various types of stress, the hairstyle has a tendency to take on an unwanted permanent shape that cannot be readily modified.

Cosmetic compositions for maintaining and/or setting the hairstyle that provide the head of hair with, a long-lasting set, good cosmetic properties, such as properties of conditioning the head of hair that confer successful untangling, softness and a pleasant, non-tacky appearance, and also ease of use and low risks when used, are therefore sought.

Moreover, cleansing or makeup-removing products having cleansing and/or makeup-removing properties and care and/or conditioning properties are also sought for the skin, for example human facial skin, and the skin appendages, for example eyelashes and eyebrows.

In addition, whether for the head of hair or the skin, cosmetic compositions capable of forming, on these keratin materials, a flexible film that follows the movements of the skin or of the head of hair, without any effect of tautness or of heaviness or any feeling of rigidity, are also sought.

The present inventors have discovered that it is possible to remedy at least one of the technical problems mentioned above using polymers defined herein in a rinse-out composition.

One aspect of the present disclosure is thus a rinse-out cosmetic composition comprising, in a cosmetically acceptable medium, at least one elastomeric film-forming polymer, wherein the film obtained by drying at least one elastomeric film-forming polymer at ambient temperature (22° C.±2° C.) and at a relative humidity of 55%±5%, has properties comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%,
wherein the at least one elastomeric film-forming polymer is water-soluble.

This composition makes it possible, for example, to obtain a flexible film that is not brittle on human keratin materials and that entirely follows their movements.

For the purpose of the present disclosure, the term “at least one” elastomeric film-forming polymer means one or more (2, 3 or more) elastomeric film-forming polymers.

The composition according to the present disclosure, for example, may provide human keratin materials, for example the hair, with conditioning, and can also provide the hair with a very high level (highly set) styling effect and an excellent hold of the hairstyle over time. The composition according to the present disclosure may, for example, be a rinse-out hair composition, including, for example, shampoos and conditioners (or conditioning compositions) that can be applied after dyeing, bleaching, relaxing or permanent-waving of the hair.

The hair treated with the compositions according to the present disclosure may exhibit very good cosmetic properties without any charge effect.

For the purpose of the present disclosure, the term “rinse-out composition” means any composition that is formulated so as to be rinsed out after application to human keratin materials such as the hair or the skin.

The rinse-out composition may be in any of the conventional forms of rinse-out cosmetic compositions, for example in the form of lotions, mousses or milks, and may represent, but without being limited thereto, shampoos, conditioners, lotions for rinsing the hair, permanent-waving compositions, waving compositions, compositions for dyeing the hair, products for use before or after a hair-dyeing treatment, products for use before or after a permanent-waving treatment, relaxing compositions, products to be used before or after a relaxing treatment, and combinations thereof, body hygiene products such as cleansing products for the body or for the face, or makeup-removing products, for example for the skin of the face or of the neck or for the eyes, in the form of a lotion or of a milk.

For example, the composition of the present disclosure may be a hair shampoo that has a hair cleansing effect and can also have a conditioning effect. A conditioner is a product that has a conditioning effect on the hair without a cleansing and/or styling effect.

Another embodiment of the present disclosure relates to a method for shaping or maintaining the hairstyle, comprising the use of a cosmetic composition as defined herein on keratin materials, for example the hair, the application being followed by rinsing with water and/or with an aqueous composition.

Another embodiment of the present disclosure relates to a cosmetic method for cleansing and/or for removing makeup from the skin, such as the scalp, and/or human keratin fibers, such as the hair, the eyelashes and the eyebrows, comprising the application of a cosmetic composition as defined herein, to the skin and/or said human keratin fibers, this application being followed by rinsing with water and/or with an aqueous composition.

Another embodiment of the present disclosure relates to a cosmetic method for conditioning human keratin materials, and for example the skin, such as the scalp, and/or human keratin fibers, comprising the application of a cosmetic composition as defined herein, to these human keratin materials, this application being followed by rinsing with water and/or with an aqueous composition.

Yet another embodiment subject of the present disclosure relates to the use of the cosmetic composition as defined herein for producing cosmetic compositions intended both for the cleansing and for the care of human keratin materials, such as the skin, the hair, the eyelashes or the nails. A further embodiment of the present disclosure relates to the use of the cosmetic composition as defined herein for producing a cosmetic hair product for the purpose of maintaining and/or setting the hairstyle.

In certain embodiments, the composition of the present disclosure is in the form of a shampoo.

Thus, one embodiment of the present disclosure relates to a hair shampoo comprising, in a cosmetically acceptable medium, at least one washing base and at least one elastomeric film-forming polymer as defined herein.

For the purpose of the present disclosure, the expression “film obtained by drying at ambient temperature (22° C.±2° C.) and at a relative humidity of 55%±5%” is intended to mean the film obtained, under these conditions, from a mixture containing 6% of active material (a.m.) of elastomeric film-forming polymer in a mixture of 30% by weight of ethanol and 70% by weight of water, relative to the total alcohol+water weight, the amount of mixture being adjusted so as to obtain, in a Teflon matrix, a film 500 μm±50 μm thick. The drying is continued until the weight of the film no longer changes, which represents approximately 12 days. The at least one film-forming polymers that are soluble or partially soluble in ethanol are tested in ethanol alone. The other polymers are tested in water alone, in soluble or dispersed form.

For the purpose of the present disclosure, the elongation at break and the recovery rate are evaluated by means of the tests described below.

To carry out the tensile tests, the film is cut up into rectangle-shaped test pieces, 80 mm long and 15 mm wide.

The tests are carried out on a device sold under the name Lloyd or sold under the name Zwick, under the same temperature and humidity conditions as for the drying, i.e. a temperature of 22° C.±2° C. and a relative humidity of 50%±5%.

The test pieces are drawn at a speed of 20 mm/min and the distance between the jaws is 50±1 mm.

To determine the instantaneous recovery (Rᵢ), the following procedure is carried out:
the test piece is drawn by 150% (εₘₐₓ), i.e. 1.5 times its initial length (l₀),
the stress is released by applying a return speed equal to the tensile speed, i.e. 20 mm/min, and the percentage elongation of the test piece is measured, after a return to zero strain (εᵢ).

The % instantaneous recovery (Rᵢ) is given by the formula below:
Rᵢ=((εmax−εᵢ)/εmax)×100.

To determine the recovery at 300 seconds, the test piece, having been subjected to the above operations, is maintained at zero stress for a further 300 seconds, and its percentage elongation (ε₃₀₀ₛ) is measured.

The % recovery at 300 seconds (R₃₀₀ₛ) is given by the formula below:
R₃₀₀ₛ=((εmax−ε₃₀₀ₛ)/εmax)×100.

For example, the at least one elastomeric polymer of the composition according to the present disclosure, may optionally be combined with a plasticizer and/or an additional film-forming agent, such that they form, under the conditions of the tests above, a film having an elongation at break ranging from 800% to 3000%; having an instantaneous recovery of 75% to 100%; and a recovery at 300 seconds ranging from 85% to 100%.

In the compositions in accordance with the present disclosure, the elastomeric film-forming polymer or the blend of elastomeric film-forming polymers may, for example, be present at a concentration ranging from 0.05% to 20% by weight, such as from 0.1% to 15% by weight, for example from 0.25% to 10% by weight, relative to the total weight of the composition.

The at least one elastomeric film-forming polymer may be chosen from polyurethanes, polyvinyl alcohols, polymers comprising at least one (meth)acrylic unit, and combinations thereof. The at least one elastomeric film-forming polymer may be in the form of a homopolymer or of a copolymer. The at least one elastomeric film-forming polymer may be in a non-crosslinked form in the composition.

The composition may, if necessary, also further comprise a plasticizer and/or at least one agent for facilitating the formation of a film of the at least one elastomeric polymer on the keratin materials, the function of which is to modify the properties of the at least one elastomeric polymer. Such an additional film-forming agent may be chosen from any of the compounds known to those skilled in the art as being capable of performing the desired function, and may, for example, be chosen from plasticizers and coalescence agents. The at least one elastomeric film-forming polymer, optionally combined with a plasticizer and/or an agent for facilitating film formation, may be capable of forming a film, after evaporation of the cosmetic medium. This evaporation may be carried out in the open air or by providing heat, for example using a dryer.

As non-limiting examples of plasticizers and/or additional agents for facilitating film formation on keratin materials, mention may be made of those described in document FR-A-2 782 917. For example, the plasticizers and/or agents may be chosen from the usual plasticizers or coalescence agents, such as:
glycols and their derivatives, such as diethylene glycol ethyl ether, diethylene glycol methyl ether, diethylene glycol butyl ether or else diethylene glycol hexyl ether, ethylene glycol ethyl ether, ethylene glycol butyl ether, ethylene glycol hexyl ether or pentylene glycol,
glyceryl esters,
propylene glycol derivatives, and for example propylene glycol phenyl ether, propylene glycol diacetate, propylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether,
acid esters, for example carboxylic acid esters, such as citrates, phthalates, adipates, carbonates, tartrates, phosphates or sebacates,
mixtures thereof.

The amount of plasticizers and/or film-forming agents may be chosen by those skilled in the art based on their general knowledge, so as to obtain a polymeric system (elastomeric polymers+plasticizer and/or additional film-forming agent) that produces a film having the desired mechanical properties, while at the same time allowing the composition to conserve the desired cosmetic properties. In practice, the amount of plasticizer and/or additional film-forming agent ranges from 0.01% to 25% by weight of the total weight of the composition, such as from 0.01% to 15%.

The composition according to the present disclosure also comprises a cosmetically acceptable medium. For purposes of the present disclosure, the term “cosmetically acceptable medium” means a non-toxic medium that may be applied to the scalp and the hair of human beings, and that may be sold without medical prescription.

In certain embodiments, the cosmetically acceptable medium of the composition of the present disclosure comprises a hydrophilic phase containing water and/or one or more water-miscible cosmetically acceptable solvents, such as C₁-C₄ monoalcohols and polyols, for instance glycerol, diglycerol, propylene glycol, diethylene glycol, sorbitol, or glycol ethers. For example, the cosmetically acceptable medium of the composition comprises water or one or more cosmetically acceptable solvents such as alcohols or water-solvent(s) mixtures, and this or these solvent(s) may be chosen from C₁-C₄ alcohols. Among these alcohols, mention may be made of ethanol, isopropanol or t-butanol. In certain embodiments, ethanol may be used.

In one embodiment, the at least one elastomeric film-forming polymer of the present disclosure is water-soluble or water-dispersible. For example, the at least one elastomeric film-forming polymer(s) may be soluble at at least 10 g of active material in 90 g of water, at ambient temperature and atmospheric pressure, the solution being transparent to the naked eye.

The film formed may have a low water sensitivity, for example in an atmosphere with a relative humidity of 30% to 80%, i.e. the film keeps its elastomeric properties for several hours. It may be flexible and non-brittle, and correctly follows the movements of the skin and/or of the scalp. For example, between 30% and 80% relative humidity, the elongation at break of the film obtained does not vary by more than 50% (±400%) and/or its instantaneous recovery does not vary by more than 25% (18.75%); in other words, between 30% and 80% relative humidity, the elongation at break of the film obtained may be between 400% and 1200% and/or its instantaneous recovery may be between 57% and 93%.

In other embodiments, the cosmetically acceptable medium of the composition may also comprise an oily phase comprising one or more fatty substances that are liquid at ambient temperature (22° C.) and atmospheric pressure, and water-immiscible, called “oils”. These oils may, for example, be makeup-removing oils: they may for example be of synthetic origin, such as fatty acid esters or else linear or cyclic silicones, that may or may not be modified, or oils of mineral or plant origin, it being possible for these oils to be volatile or non-volatile.

The composition of the present disclosure may also comprise at least one surfactant, this surfactant being chosen from non-ionic surfactants, anionic surfactants, cationic surfactants, amphoteric surfactants, and mixtures thereof. In certain embodiments, the composition of the present disclosure may comprise at least one anionic surfactant and at least one amphoteric surfactant.

The composition of the present disclosure may be in the form of a hair shampoo. Thus, the composition according to the present disclosure may also comprise a washing base comprising at least one surfactant chosen from anionic, non-ionic or amphoteric surfactants, and mixtures thereof.

As an anionic surfactant that may be used in the present disclosure, mention may, for example, be made of salts, for example alkali metal salts such as sodium salts, ammonium salts, amine salts, amino alcohol salts or alkaline earth metal salts, for example magnesium salts, of the following types:
alkyl sulphates, alkyl ether sulphates, alkylamido ether sulphates, alkylaryl polyether sulphates, monoglyceride sulphates; alkylsulphonates, alkylamidesulphonates, alkylarylsulphonates, α-olefin sulphonates, paraffin sulphonates, alkylsulphosuccinates, alkyl ether sulphosuccinates, alkylamide sulphosuccinates, alkylsulphoacetates, acyl-sarcosinates and acylglutamates, the alkyl and acyl groups of all these compounds containing from 6 to 24 carbon atoms and the aryl group may denote a phenyl or benzyl group, and mixtures thereof.

It is also possible to use, as an anionic surfactant, C₆-C₂₄ alkyl monoesters of polyglycoside dicarboxylic acids such as alkyl glycoside citrates, polyalkyl glycoside tartrates and polyalkyl glycoside sulphosuccinates, alkylsulphosuccinamates, acyl-isethionates and N-acyltaurates, the alkyl and the acyl group of all these compounds containing from 12 to 20 carbon atoms, and mixtures thereof.

Another group of anionic surfactants that may be used in the composition of the present disclosure is acyllactylates in which the acyl group contains from 8 to 20 carbon atoms.

In addition, mention may also be made of alkyl-D-galactosideuronic acids and the salts thereof, and also polyoxyalkylenated (C₆₋₂₄)alkyl ether carboxylic acids, polyoxyalkylenated (C₆₋₂₄)alkyl(C₆₋₂₄)aryl ether carboxylic acids, polyoxyalkylenated (C₆₋₂₄)alkylamido ether carboxylic acids, and salts thereof, for example those containing from 2 to 50 ethylene oxide units, and mixtures thereof.

Examples of anionic surfactants that may be used include alkyl sulphates, alkyl ether sulphates and alkyl ether carboxylates, and mixtures thereof, for example in the form of alkali metal salts or alkaline earth metal salts, ammonium salts, amine salts or amino alcohol salts.

As an amphoteric surfactant that may be used in the present disclosure, mention may be made of derivatives of secondary or tertiary aliphatic amines, in which the aliphatic group is a linear or branched chain containing from 8 to 22 carbon atoms and containing at least one anionic group such as, for example, a carboxylate, sulphonate, sulphate, phosphate or phosphonate group. Mention may for example be made of (C₈₋₂₀)alkylbetaines, sulphobetaines, (C₈₋₂₀)alkylamido(C₆₋₈)alkylbetaines, (C₈₋₂₀)alkylamido(C₆₋₈)alkylsulphobetaines, and mixtures thereof.

Among the amine derivatives, mention may be made of the products sold under the name Miranol®, as described in U.S. Pat. Nos. 2,528,378 and 2,781,354 and classified in the CTFA dictionary, 3rd edition, 1982, under the names Amphocarboxyglycinate and Amphocarboxypropionate, having the respective structures (1) and (2):
Rₐ—CONHCH₂CH₂—N(R_{b})(R_{c})(CH₂COO) (1)
in which:
Rₐ represents an alkyl group derived from an acid Rₐ-COOH present in hydrolysed coconut oil, a heptyl group, a nonyl group or an undecyl group,
R_{b} represents a beta-hydroxyethyl group, and
R_{c} represents a carboxymethyl group; and
   R′ₐ—CONHCH₂CH₂—N(B)(C) (2)
   in which:
B represents —CH₂CH₂OX′,
C represents (CH₂)ₙ—Y′, with z=1 or 2,
X′ represents the group —CH₂CH₂—COOH or a hydrogen atom,
Y′ represents —COOH or the group —CH₂—CHOH—SO₃H,
R′ₐ represents an alkyl group of an acid R′a-COOH present in coconut oil or in hydrolysed linseed oil, an alkyl group, for example a C₁₋₇ alkyl group and its iso form, an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylampho dipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M concentrate.

Among examples of the amphoteric surfactants that may be used, (C₈₋₂₀)alkylbetaines, (C₈₋₂₀)alkylamido(C₆₋₈)alkylbetaines, alkylamphodiacetates, and mixtures thereof are included.

As a non-ionic surfactant that may be used in the composition according to the present disclosure, mention may be made of the known compounds described for example in the book “Handbook of Surfactants” by M. R. Porter, published by Blackie & Son (Glasgow and London), 1991, pp 116-178). They are chosen, for example, from alcohols, alpha-diols, (C₁₋₂₀)alkylphenols, or polyethoxylated, polypropoxylated or polyglycerolated fatty acids having a fatty chain containing, for example, from 8 to 18 carbon atoms, the number of ethylene oxide or propylene oxide groups possibly ranging for example from 2 to 50 and the number of glycerol groups possibly ranging for example from 2 to 30, and mixtures thereof.

Mention may also be made, as non-ionic surfactants that may be used in the present disclosure, of condensates of ethylene oxide and of propylene oxide with fatty alcohols; polyethoxylated fatty amides having, for example, from 2 to 30 ethylene oxide units; polyglycerolated fatty amides containing, on average, from 1 to 5 glycerol groups, and for example from 1.5 to 4; ethoxylated fatty acid esters of sorbitan having from 2 to 30 ethylene oxide units; fatty acid esters of sucrose; fatty acid esters of polyethylene glycol; (C₆₋₂₄)alkylpolyglycosides; N—(C₆₋₂₄)alkylglucamine derivatives; amine oxides such as (C₁₀₋₁₄)alkylamine oxides or N—(C₁₀₋₁₄)acylaminopropylmorpholine oxides; and mixtures thereof.

Among examples of the non-ionic surfactants mentioned above, (C₆₋₂₄)alkylpolyglycosides may be used in certain embodiments.

According to another embodiment of the present disclosure, the composition comprises at least one washing base comprising at least one anionic surfactant and, optionally, at least one amphoteric or non-ionic surfactant.

The total amount of surfactant in the composition described herein may, for example, generally range from 0.01% to 50% by weight, such as from 0.1% to 25% by weight, relative to the total weight of the composition.

For example, when the composition of the present disclosure is in the form of a shampoo, the total amount of surfactants (or washing base) may, for example, range from 4% to 50% by weight, for example from 8% to 25% by weight, relative to the total weight of the cosmetic composition.

The compositions of the present disclosure may also be in the form of a rinse-out conditioner, of compositions for permanent-waving, relaxing, dyeing or bleaching, or else in the form of rinse-out compositions to be applied before or after dyeing, bleaching, permanent-waving or relaxing, or else between the two steps of a permanent-wave or of a relaxing process.

When the composition is in the form of a rinse-out conditioner, it may comprise at least one cationic surfactant, the concentration of which may range from 0.1 and 10% by weight, such as from 0.5 to 5% by weight, relative to the total weight of the composition.

Among the cationic surfactants, mention may, for example, be made of (non-limiting list): primary, secondary or tertiary fatty amine salts, optionally polyoxyalkylenated; quaternary ammonium salts, including alkylpyridinium salts, imidazoline ammonium salts, quaternary diammonium salts, and ammonium salts containing at least one ester function.

By way of quaternary ammonium salts, mention may, for example, be made of:
those that have general formula (V) below: 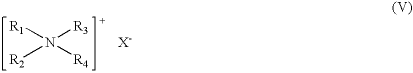
in which the symbols R₁ to R₄, which may be identical of different, represent a linear or branched aliphatic radical containing from 1 to 30 carbon atoms, or an aromatic radical such as aryl or alkylaryl. The aliphatic radicals may contain hetero atoms such as for example oxygen, nitrogen or sulphur, and halogens. The aliphatic radicals may, for example, be chosen from the following radicals: alkyl, for example C₁₂-C₂₂ alkyl, alkoxy, polyoxy(C₂-C₆)alkylene, alkylamide, (C₁₂-C₂₂)alkylamido(C₂-C₆)alkyl, (C₁₂-C₂₂)alkyl acetate, hydroxyalkyl, containing approximately from 1 to 30 carbon atoms; X— may be an anion chosen from halides, phosphates, acetates, lactates, (C₂-C₆)alkyl sulphates, alkylsulphonates or alkylarylsulphonates;
quaternary ammonium salts of imidazoline such as, for example, those of formula (VI) below: 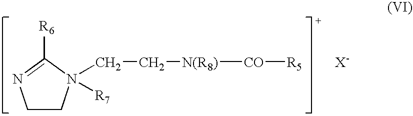
in which R₅ represents an alkenyl or alkyl radical containing from 8 to 30 carbon atoms, for example derived from tallow or coconut fatty acids, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, X is an anion chosen from the group of halides, phosphates, acetates, lactates, alkyl sulphates, alkylsulphonates or alkylarylsulphonates. In one embodiment, R₅ and R₆ denote a mixture of alkenyl or alkyl radicals containing from 12 to 21 carbon atoms, for example derived from tallow fatty acids, R₇ denotes methyl, R₈ denotes hydrogen. Such a product is, for example, quaternium-27 (CTFA 1997) or quaternium-83 (CTFA 1997) sold under the names “Rewoquat®” W75, W90, W75PG or W75HPG by the company Witco;
quaternary diammonium salts of formula (VII): 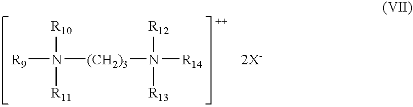
in which R₉ denotes an aliphatic radical containing approximately from 16 to 30 carbon atoms, R₁₀, R₁₁, R₁₂, R₁₃ and R₁₄, which may be identical or different, are chosen from hydrogen or an alkyl radical containing from 1 to 4 carbon atoms, and X— may be an anion chosen from halides, acetates, phosphates, nitrates, ethyl sulphates and methyl sulphates. Such quaternary diammonium salts comprise for example propane tallow diammonium dichloride;
quaternary ammonium salts comprising at least one ester function, such as those of formula (VIII) below: 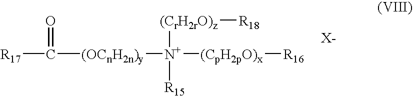
   in which:
R₁₅ may be chosen from C₁-C₆ alkyl radicals and C₁-C₆ hydroxyalkyl or dihydroxyalkyl radicals;
R₁₆ may be chosen from:
the radical 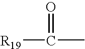
linear or branched, saturated or unsaturated C₁-C₂₂ hydrocarbon-based radicals R₂₀, and
a hydrogen atom,
   R₁₇ may be chosen from:
the radical 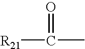
linear or branched, saturated or unsaturated C₁-C₆ hydrocarbon-based radicals R₂₂, and
a hydrogen atom,
R₁₇, R₁₉ and R₂₁, which may be identical or different, may be chosen from linear or branched, saturated or unsaturated C₇-C₂₁ hydrocarbon-based radicals;
r, n and p, which may be identical or different, may be integers ranging from 2 to 6;
y may be an integer ranging from 1 to 10;
x and z, which may be identical or different, may be integers ranging from 0 to 10;
X— may be a simple or complex, organic or inorganic anion;
with the proviso that the sum of x+y+z may be from 1 to 15, that when x is 0, then R₁₆ denotes R₂₀, and that when z is 0, then R₁₈ denotes R₂₂.

The R₁₅ alkyl radicals may be linear or branched, and in certain embodiments is linear. R₁₅ may denote a methyl, ethyl, hydroxyethyl or dihydroxypropyl radical, and in certain embodiments may be a methyl or ethyl radical.

In certain embodiments, the sum of x+y+z may be from 1 to 10.

When R₁₆ is a hydrocarbon-based radical R₂₀, it may be long and contain from 12 to 22 carbon atoms, or short and contain from 1 to 3 carbon atoms.

When R₁₈ is a hydrocarbon-based radical R₂₂, it may contains 1 to 3 carbon atoms.

In certain embodiments, R₁₇, R₁₉ and R₂₁, which may be identical or different, may be chosen from linear or branched, saturated or unsaturated C₁₁-C₂₁ hydrocarbon-based radicals, such as linear or branched, saturated or unsaturated C₁₁-C₂₁ alkyl and alkenyl radicals.

In certain embodiments, x and z, which may be identical or different, may be 0 or 1. In certain embodiments, y may be equal to 1.

In certain embodiments, r, n and p, which may be identical or different, may be 2 or 3, such as equal to 2.

The anion X— may be a halide (chloride, bromide or iodide) or a (C₁-C₄)alkyl sulphate, such as methyl sulphate. However, methanesulphonate, phosphate, nitrate, tosylate, an anion derived from an organic acid, such as acetate or lactate, or any other anion that is compatible with the ammonium containing ester function, may be used.

In certain embodiments, the anion X— may be chloride or methyl sulphate.

Exemplary ammonium salts may be used in the composition according to the present disclosure include those of formula (VIII) in which:
R₁₅ denotes a methyl or ethyl radical;
x and y are equal to 1;
z is equal to 0 or 1;
r, n and p are equal to 2;
R₁₆ is chosen from:
   the radical 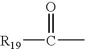
   methyl, ethyl or C₁₄-C₂₂ hydrocarbon-based radicals;
   a hydrogen atom;
R₁₈ is chosen from:
   the radical 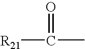
   a hydrogen atom;
R₁₇, R₁₉ and R₂₁, which may be identical or different, may be chosen from linear or branched, saturated or unsaturated C₁₃-C₁₇ hydrocarbon-based radicals, such as linear or branched, saturated or unsaturated C₁₃-C₁₇ alkyl and alkenyl radicals. In certain embodiments the hydrocarbon-based radicals may be linear.

Examples that may be mentioned include the compounds of formula (VIII) such as the diacyloxyethyldimethylammonium, diacyloxyethylhydroxyethylmethyl ammonium, monoacyloxyethyldihydroxyethylmethylammonium, triacyloxyethylmethylammonium and monoacyloxyethylhydroxyethyldimethylammonium salts (and for example chloride or methyl sulphate), and mixtures thereof. The acyl radicals may contain 14 to 18 carbon atoms and may be obtained from a plant oil such as palm oil or sunflower oil. When the compound contains several acyl radicals, these radicals may be identical or different.

These products may be obtained, for example, by direct esterification of triethanolamine, triisopropanolamine, an alkyldiethanolamine or an alkyldiisopropanolamine, which are optionally oxyalkylenated, with fatty acids or with mixtures of fatty acids of plant or animal origin, or by transesterification of the methyl esters thereof. This esterification may be followed by a quaternization using an alkylating agent such as an alkyl halide (for example a methyl or ethyl halide), a dialkyl sulphate (for example dimethyl or diethyl sulphate), methyl methanesulphonate, methyl para-toluenesulphonate, glycol chlorohydrin or glycerol chlorohydrin.

Such compounds are sold, for example, under the names Dehyquart® by the company Cognis, Stepanquat® by the company Stepan, Noxamium® by the company Ceca or Rewoquat® WE 18 by the company Rewo-Goldschmidt.

The composition according to the present disclosure may comprise a mixture of quaternary ammonium monoester, diester and triester salts, with a majority by weight of diester salts.

As a mixture of ammonium salts, use may, for example, be made of the mixture containing 15 to 30% by weight of acyloxyethyldihydroxyethylmethylammonium methyl sulphate, 45 to 60% of diacyloxyethylhydroxyethylmethylammonium methyl sulphate and 15 to 30% of triacyloxyethylmethylammonium methyl sulphate, the acyl radicals containing from 14 to 18 carbon atoms and being obtained from palm oil, that is optionally partially hydrogenated.

Use may also be made of the ammonium salts comprising at least one ester function, described, for example, in U.S. Pat. Nos. 4,874,554 and 4,137,180.

Among the quaternary ammonium salts mentioned above, use may be made of those corresponding to formula (V). Mention may, for example, be made, of dipalmitoylethylhydroxyethylmethylammonium salts (and for example methosulphates), tetraalkylammonium salts (and for example chlorides) such as, for example, dialkyldimethylammonium or alkyltrimethylammonium salts (and for example chlorides), in which the alkyl radical contains from approximately 12 to 22 carbon atoms, for example behenyltrimethylammonium, distearyldimethylammonium, cetyltrimethylammonium or benzyldimethylstearylammonium salts (and for example chlorides), palmitylamidopropyltrimethylammonium salts (and for example chlorides) or stearamidopropyldimethyl(myristyl acetate)ammonium salts (and for example chlorides), and for example the stearamidopropyldimethyl(myristyl acetate)ammonium chloride salt under the name Ceraphyl® 70 by the company Van Dyk.

Exemplary cationic surfactants that may be used in the composition of the present disclosure include those chosen from quaternary ammonium salts, and for example from behenyltrimethylammonium chloride, dipalmitoylethylhydroxyethylmethylammonium methosulphate, cetyltrimethylammonium chloride, quaternium-83, behenylamidopropyl-2,3-dihydroxypropyldimethylammonium chloride and palmitylamidopropyltrimethylammonium chloride.

The composition of the present disclosure may also contain at least one additional ingredient conventionally used in the cosmetics field, and chosen from formulation additives and cosmetic active ingredients that have a beneficial effect on keratin materials such as the skin and keratin fibers such as the hair.

As formulation additives, mention may be made of aqueous phase or fatty phase thickeners, anionic, non-ionic, cationic or amphoteric, fixing or non-fixing polymers, waxes, gums, fillers, fragrances, preserving agents, sequestering agents, pH modifiers such as basic solutions (for example, solutions of sodium hydroxide, of potassium hydroxide or of ammonium hydroxide, aminomethylpropanols and primary, secondary and tertiary amines) or buffer solutions (for example, sodium bicarbonate), or else acid solutions (for example, solutions of citric acid or hydrochloric acid), pearlescent agents, dyestuffs and dye precursors, opacifiers and any other additive conventionally used in cosmetic compositions intended to be applied to keratin materials such as the skin, the hair or the eyelashes.

The composition of the present disclosure may also contain at least one cosmetic active agent that has a beneficial effect, such as zinc salts of an organic acid (acetate, glycolate, lactate, gluconate or citrate) or of an inorganic acid (chloride and sulphate), sunscreens, proteins, vitamins (A, E, B₂, B₅, F, C), provitamins, ceramides, pseudoceramides, free-radical scavengers, or plant extracts.

The amounts of the various additional ingredients of the composition according to the present disclosure may be those generally used in the fields under consideration, and may be, for example, within the range of from 0.001% to 20% of the total weight of the composition. In addition, this composition is prepared according to the usual methods.

These compositions may be packaged in various forms, for example in bottles, that are optionally provided with a pump, heating bags, tubes, or simple pots for taking up the composition by hand. The compositions in accordance with the present disclosure may be in the form of a cream, a gel, a water-in-oil or oil-in-water emulsion, a lotion, a spray, a mousse or a wax.

In one embodiment, the pH of the aqueous phase may range from 2 to 11, such as from 3 to 10, for example from 5 to 8.

Of course, those skilled in the art will take care to choose the optional compound(s) to be added to the composition according to the present disclosure in such a way that the advantageous properties intrinsically associated with the composition in accordance with the present disclosure are not, or are not substantially, impaired by the envisaged addition.

The compositions in accordance with the present disclosure may be applied to keratin materials such as the hair, the skin and the eyelashes, in the dry or wet state.

Other than in the examples, or where otherwise indicated, all numbers expressing quantities of ingredients, reaction conditions, and so forth used in the specification and claims are to be understood as being modified in all instance by the term “about.” Accordingly, unless indicated to the contrary, the numerical parameters set forth in the following specification and attached claims are approximations that may vary depending upon desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contain certain errors necessarily resulting from the standard deviation found in their respective testing measurements. By way of non-limiting illustration, a concrete example of preparation of the composition according to the present disclosure are given below.

The amounts are given as percentage by mass, and A.M. signifies active material.

### Prophetic Example

### Shampoo Conditioner

The following composition can be prepared by simply mixing the various constituents under cold conditions (ambient temperature).

| Sodium lauryl ether sulphate (2.2 EO)* | 15.4 | AM |
|---|---|---|
| Sodium cocoamphocarboxyglycinate | 3 | AM |
| (Miranol C2M from Rhodia Chimie | | |
| Polyurethane (NMDEA¹⁾/PTMO 2900²⁾/IPDI³⁾ - 3/1/4) | 1.5 | AM |
| Preserving agent | q.s. | |
| pH adjusted to | 7 | |

| | |
|---|---|
| Water | qs 100% |

*EO signifies ethylene oxide
¹⁾N-methyldiethanolamine
²⁾poly(tetramethylene oxide) having a weight-average molecular mass of 2900
³⁾isophorone diisocyanate

The polyurethane copolymer can be prepared as described in document FR-A-2 815 350, and for example according to the method for synthesizing the polyurethane PU2 of Example 1, replacing the poly(tetramethylene oxide) having a weightaverage molecular mass of 1400 with a poly(tetramethylene oxide) having a weightaverage molecular mass of 2900.

This polyurethane copolymer can have the following characteristics, measured as above:
ε_{b}=1500%;
Rᵢ=82%;
R₃₀₀=92%; and
water-soluble at at least 10 g per litre.

This shampoo can provide effective washing of the hair and can provide the hair with good conditioning properties such as untangling and smoothing, and also a good styling effect (good degree of setting and very good hold of the hairstyle over time), that follows the movements of the head of hair.

What is claimed is:

1. A rinse-out cosmetic composition comprising, in a cosmetically acceptable medium,
at least one elastomeric film-forming polymer, wherein the film obtained by drying the at least one elastomeric film-forming polymer at ambient temperature and at a relative humidity of 55%±5%, has properties comprising:
(a) an elongation at break (ε_{b}) of greater than or equal to 800%;
(b) an instantaneous recovery (Rᵢ) at least equal to 75%, after an elongation of 150%; and
(c) a recovery (R₃₀₀) at 300 seconds of greater than 80%,
wherein the at least one elastomeric film-forming polymer is water-soluble.

2. A composition according to claim 1, wherein the composition is in the form of a cleansing composition.

3. A composition according to claim 1, wherein the composition comprises at least one surfactant.

4. A composition according to claim 3, wherein the total amount of the at least one surfactant ranges from 0.01% to 50% by weight, relative to the total weight of the composition.

5. A composition according to claim 4, wherein the total amount of the at least one surfactant ranges from 0.1% to 25% by weight relative to the total weight of the composition.

6. A composition according to claim 1, wherein the composition comprises at least one washing base comprising at least one surfactant chosen from anionic surfactants, non-ionic surfactants and amphoteric surfactants.

7. A composition according to claim 1, wherein the composition comprises at least one washing base comprising at least one anionic surfactant and at least one amphoteric surfactant.

8. A composition according to claim 6, wherein the at least one washing base is present in an amount ranging from 4% to 50% by weight relative to the total weight of the composition.

9. A composition according to claim 8, wherein the at least one washing base is present in an amount ranging from 8% to 25% by weight relative to the total weight of the composition.

10. A composition according to claim 1, wherein the composition is in the form of a hair shampoo.

11. A composition according to claim 1, wherein the composition is in the form of a hair product that maintains and/or sets the hair.

12. A composition according to claim 1, wherein the at least one elastomeric film-forming polymer is present in an amount ranging from 0.05% to 20% by weight, relative to the total weight of the composition.

13. A composition according to claim 12 wherein the at least one elastomeric film-forming polymer is present in an amount ranging from 0.1% to 15% by weight, relative to the total weight of the composition.

14. A composition according to claim 13, wherein the at least one elastomeric film-forming polymer is present in an amount ranging from 0.25% to 10% by weight, relative to the total weight of the composition.

15. A composition according to claim 1, wherein the at least one elastomeric film-forming polymer is chosen from polyurethanes, polyvinyl alcohols, and polymers comprising at least one (meth)acrylic unit.

16. A composition according to claim 1, wherein the cosmetically acceptable medium comprises a hydrophilic phase comprising water and/or at least one water-miscible cosmetically acceptable solvent.

17. A composition according to claim 16 wherein the at least one water-miscible cosmetically acceptable solvent is chosen from C₁-C₄ alcohols.

18. A composition according to claim 1, wherein the composition comprises at least one additional ingredient chosen from cosmetic active ingredients and formulation additives.

19. A method for conditioning human keratin materials comprising
(i) applying to the human keratin materials a cosmetic composition comprising, in a cosmetically acceptable medium, at least one elastomeric film-forming polymer, wherein the film obtained by drying the at least one elastomeric film-forming polymer at ambient temperature and at a relative humidity of 55%±5%, has properties comprising:
(a) an elongation at break (ε_{b}) of greater than or equal to 800%;
(b) an instantaneous recovery (Rᵢ) at least equal to 75%, after an elongation of 150%; and
(c) a recovery (R₃₀₀) at 300 seconds of greater than 80%,
wherein the at least one elastomeric film-forming polymer is water-soluble, and
(ii) rinsing the cosmetic composition applied to the human keratin materials with water and/or an aqueous composition.

20. A method for cleansing and/or for removing makeup from the skin and/or human keratin fibers comprising
(i) applying to the skin and/or human keratin fibers a cosmetic composition comprising, in a cosmetically acceptable medium, at least one elastomeric film-forming polymer, wherein the film obtained by drying the at least one elastomeric film-forming polymer at ambient temperature and at a relative humidity of 55%±5%, has properties comprising:
(a) an elongation at break (ε_{b}) of greater than or equal to 800%;
(b) an instantaneous recovery (Rᵢ) at least equal to 75%, after an elongation of 150%; and
(c) a recovery (R₃₀₀) at 300 seconds of greater than 80%,
wherein the at least one elastomeric film-forming polymer is water-soluble, and
(ii) rinsing the cosmetic composition applied to the skin and/or human keratin fibres with water and/or an aqueous composition.

21. A method according to claim 20, wherein the skin is present on a scalp.

22. A method according to claim 20, wherein skin, hair, eyelashes and/or nails are cleansed.

23. A method for maintaining or shaping a hairstyle, comprising
(i) applying to the hair a cosmetic composition comprising, in a cosmetically acceptable medium, at least one elastomeric film-forming polymer, wherein the film obtained by drying the at least one elastomeric film-forming polymer at ambient temperature and at a relative humidity of 55%±5%, has a mechanical profile comprising:
(a) an elongation at break (ε_{b}) of greater than or equal to 800%;
(b) an instantaneous recovery (Rᵢ) at least equal to 75%, after an elongation of 150%; and
(c) a recovery (R₃₀₀) at 300 seconds of greater than 80%,
wherein the at least one elastomeric film-forming polymer is water-soluble, and
(ii) rinsing the cosmetic composition applied to the hair with water and/or an aqueous composition.
